(19) United States
(12) Patent Application Publication
(10) Pub. No.: US 2026/0263011 A1
(43) Pub. Date: Sep. 10, 2026

(54) EEG SIGNAL PREPROCESSING DEVICE AND METHOD

(71) Applicant: NEUROGRIN INC., Seoul (KR)

(72) Inventor: MYEONG SEONG BAK, Seoul (KR)

(21) Appl. No.: 18/876,727

(22) PCT Filed: Aug. 26, 2022
(86) PCT No.: PCT/KR2022/012823

§ 371 Date: Dec. 19, 2024

(30) Foreign Application Priority Data
Jun. 29, 2022 (KR) 10-2022-0079763

(51) Int. Cl. A61B 5/00; A61B 5/374; G06N 3/0464

(52) U.S. Cl. CPC A61B 5/7267; A61B 5/374; A61B 5/7203; G06N 3/0464

(57) ABSTRACT

An electroencephalogram (EEG) signal preprocessing device includes a reference potential adjustment unit to remove a common noise component by adjusting a reference potential of an EEG signal, a filtering unit to filter a frequency band of the EEG signal, a signal conversion unit to convert the EEG signal from a time domain signal into a frequency domain signal, a machine learning model implemented with a 1D convolutional neural network in which the numbers of layers and channels of the EEG signal are the same, a machine learning model training unit to generate training data having the EEG signal as an input condition and an EEG signal from which a noise component is removed as an output condition, and perform supervised learning on the machine learning model through the training data, and a machine learning model analysis unit to obtain and output the EEG signal through the machine learning model.

### BACKGROUND

### 1. Technical Field

The present disclosure relates to an electroencephalogram (EEG) signal preprocessing device and method which may more efficiently perform an EEG signal preprocessing operation.

### 2. Background Art

This work was supported by the 2024 7th Seoul Innovation Challenge (final) Program (IC240019, Development of a physical therapy bio-healthcare system using brain-machine interface technology) through the Seoul Business Agency (SBA), funded by the Seoul Metropolitan Government.

An electroencephalogram (EEG) measurement device is a device for measuring a minute voltage change caused by brain activity from outside the skull. EEG measurement devices have been used for a long time to diagnose brain diseases such as epilepsy and sleep disorders and have the advantages of being non-invasive and portable.

EEG signals have a very high temporal resolution but a low spatial resolution, and thus, a spatial resolution is ensured by measuring signals from multiple parts of the skull. Because multiple cells in brain neurons simultaneously resonate, it is common to analyze frequency bands separately. For example, there are frequency powers such as gamma waves and beta waves.

In order to analyze high-quality frequency power from these EEG signals, a series of preprocessing processes are required, and the series of preprocessing processes include removing noise caused by eye or body movements and mechanical devices mixed into the EEG signals.

However, because the pattern of such noise is not constant, in the related art, as shown in FIG. 1, a trained person visually checks and removes noise, and then additionally removes a noise component through independent component analysis (ICA) to extract only high-quality signals.

However, this series of processes requires a lot of cost and time, and in particular, because ICA may only be performed after signal acquisition is completed, real-time analysis is impossible.

### SUMMARY

According to an aspect of the present disclosure, there is provided an electroencephalogram (EEG) signal preprocessing device and method which may reduce the cost and time required for data analysis and further enable real-time analysis by replacing independent component analysis (ICA) and data inspection processes through a machine learning model.

Also, there is provided an EEG signal preprocessing device and method which may respond to all kinds of situations by using a supervised learning method and also enable learning about numerous noises in real situations.

Objectives of the present disclosure are not limited thereto, and other unmentioned objectives will be clearly understood by one of ordinary skill in the art to which the present disclosure pertains from the following description.

To solve the problems, according to an embodiment of the present disclosure, an electroencephalogram (EEG) signal preprocessing device includes a reference potential adjustment unit configured to remove a common noise component by adjusting a reference potential of an EEG signal, a filtering unit configured to filter a frequency band of the EEG signal, a signal conversion unit configured to convert the EEG signal from a time domain signal into a frequency domain signal, a machine learning model implemented with a one-dimensional (1D) convolutional neural network (CNN) in which a number of layers is the same as a number of channels of the EEG signal, a machine learning model training unit configured to generate a plurality of training data having the EEG signal as an input condition and an EEG signal from which a noise component is removed as an output condition, and then perform supervised learning on the machine learning model through the plurality of training data, and a machine learning model analysis unit configured to, when EEG signal preprocessing is requested, obtain and output an EEG signal from which a noise component corresponding to the EEG signal is removed through the machine learning model.

The machine learning model training unit may be further configured to re-train the machine learning model by using an input signal and an output signal of the machine learning model analysis unit.

To solve the problems, according to another embodiment of the present disclosure, an electroencephalogram (EEG) signal preprocessing method includes removing a common noise component by adjusting a reference potential of an EEG signal, filtering a frequency band of the EEG signal, converting the EEG signal from a time domain signal into a frequency domain signal, generating a plurality of training data having the EEG signal as an input condition and an EEG signal from which a noise component is removed as an output condition, and then performing supervised learning on a machine learning model through the plurality of training data, and when EEG signal preprocessing is requested, obtaining and outputting an EEG signal from which a noise component corresponding to the EEG signal is removed through the machine learning model, wherein the machine learning model is implemented with a one-dimensional (1D) CNN in which a number of layers is the same as a number of channels of the EEG signal.

Because an electroencephalogram (EEG) signal preprocessing device of the present disclosure may remove a noise component included in an EEG signal by using a machine learning model, the cost and time required for data analysis may be dramatically reduced and real-time analysis may also be performed.

Also, because the machine learning model is trained by using a supervised learning method, all kinds of situations may be responded, and learning about numerous noises in real situations may also be performed.

### BRIEF DESCRIPTION OF THE DRAWINGS

FIG. 1 is a diagram for describing an electroencephalogram (EEG) signal preprocessing method according to the related art.

FIG. 2 is a diagram for describing an EEG signal preprocessing device according to an embodiment of the present disclosure.

FIG. 3 is a diagram for describing an EEG signal preprocessing method according to an embodiment of the present disclosure.

FIGS. 4 and 5 are diagrams illustrating an example of a pain analysis device using an EEG signal preprocessing device according to an embodiment of the present disclosure.

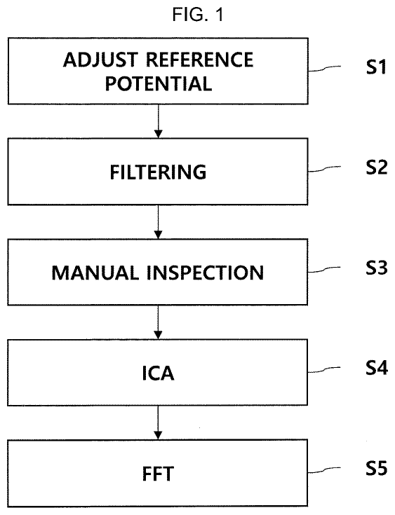

### DETAILED DESCRIPTION

The following description illustrates only a principle of the present disclosure. Therefore, one of ordinary skill in the art may implement the principle of the present disclosure and invent various devices included in the spirit and scope of the present disclosure although not clearly described or shown in the present specification. In addition, it is to be understood that all conditional terms and embodiments mentioned in the present specification are basically intended only to allow one of ordinary skill in the art to understand a concept of the present disclosure, and the present disclosure is not limited to embodiments and states particularly mentioned as such.

Further, it is to be understood that all detailed descriptions mentioning a specific exemplary embodiment of the present disclosure as well as principles, aspects, and exemplary embodiments of the present disclosure are intended to include structural and functional equivalences thereof. Further, it is to be understood that these equivalences include an equivalence that will be developed in the future as well as an equivalence that is currently well-known, that is, all devices invented so as to perform the same function regardless of a structure.

Therefore, it is to be understood that, for example, a block diagram of the present specification shows an illustrative conceptual aspect for embodying a principle of the present disclosure. Similarly, it is to be understood that all flowcharts, state transition diagrams, pseudo-code, and the like, show various processes that may be tangibly embodied in a computer-readable medium and that are executed by computers or processors regardless of whether or not the computers or the processors are clearly shown.

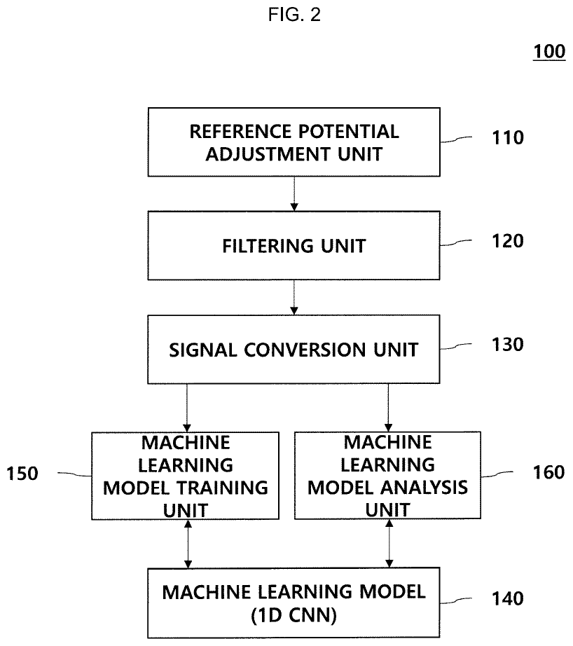

FIG. 2 is a diagram for describing an electroencephalogram (EEG) signal preprocessing device according to an embodiment of the present disclosure.

As shown in FIG. 2, an EEG signal preprocessing device 100 of the present disclosure includes a reference potential adjustment unit 110 for removing a common noise component by adjusting a reference potential of an EEG signal, a filtering unit 120 for filtering a frequency band of the EEG signal, a signal conversion unit 130 for converting the EEG signal from a time domain signal into a frequency domain signal, a machine learning model 140 implemented with a one-dimensional (1D) convolutional neural network (CNN) in which the number of layers is the same as the number of channels of the EEG signal, a machine learning model training unit 150 for generating a plurality of training data having the EEG signal as an input condition and an EEG signal from which a noise component is removed as an output condition and then performing supervised learning on the machine learning model through the plurality of training data, and a machine learning model analysis unit 160 for, when EEG signal preprocessing is requested, obtaining and outputting an EEG signal from which a noise component corresponding to the EEG signal is removed through the machine learning model.

In this case, in the machine learning model 140, a first layer includes a 1D CNN, and a kernel size of the 1D CNN is set to the same number (e.g., 55) as a frequency (fi) of each channel of the EEG signal. An operation value of a kernel that matches the frequency of each channel is then output as a vector with 1 element by summing values of all channels (ch), and because the vector is arranged as 512 parallel nodes, a matrix having a size of (1×512) per input is output (None, 1, 512).

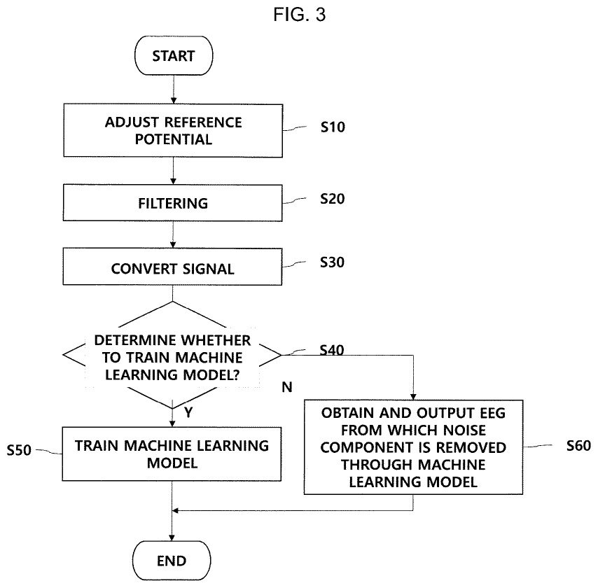

Hereinafter, an operating method of an EEG signal preprocessing device of the present disclosure will be described in more detail with reference to FIG. 3.

First, the reference potential adjustment unit 110 removes a common noise component introduced during EEG measurement by adjusting a reference potential of an EEG signal through a common average reference (CAR) method or the like (S10).

In this case, the EEG signal is a t×ch matrix signal including time t and channel number ch, and a signal value of each channel is expressed in the form of a voltage. However, because the time t ultimately uses an average value, its length is irrelevant.

For reference, the channel number ch may vary according to a used EEG measurement device, and analysis performance tends to increase as the number of channels increases, but sufficient analysis performance may be guaranteed even with a small number of channels such as 12.

The filtering unit 120 filters a frequency band of the EEG signal through a finite impulse response (FIR) filter or the like (S20).

In this case, the FIR filter is a digital filter that performs filtering with only constant (finite) values of an input signal, and has a finite length when an impulse response, which is a characteristic function of the filter, is calculated.

The FIR filter does not have a feedback component in a filter equation, and thus, when implementing the same characteristics, the order is higher than that of an IIR filter, resulting in high implementation costs (parts price, execution time, etc.). However, when phase shift (i.e., maintaining a shape of a waveform between input and output) is important, the FIR filter should be used.

The signal conversion unit 130 converts the time domain EEG signal (t×ch) into a frequency domain EEG signal (ch×fi) by using Fourier transform, wavelet transform, etc. (S30).

In more detail, the signal conversion unit 130 converts the EEG signal in the form of t×ch into a time-domain frequency signal in the form of ch×fi×t. Next, the signal conversion unit 130 obtains an average for t from data converted into ch×fi×t to obtain a result, finally converts the result into a frequency domain signal in the form of ch×fi, and outputs the frequency domain signal.

Also, the signal conversion unit 130 of the present disclosure additionally has a downsampling function to reduce a sampling rate of an input signal when necessary (e.g., reduce 1000 Hz to 300 Hz).

Also, the machine learning model training unit 150 determines whether to train the machine learning model (S40).

When it is a section where machine learning model training is performed, the machine learning model training unit 150 generates a plurality of training data having the EEG signal as an input condition and an EEG signal from which a noise component is removed as an output condition. The machine learning model training unit 150 repeatedly performs supervised learning on the machine learning model 140 to learn about a correlation between an input signal and a noise component removal result based on the plurality of training data (S50).

In this case, the machine learning model training unit 150 of the present disclosure uses a supervised learning method, which enables response to all kinds of situations and enables relearning of numerous noises (i.e., input and output signals of the machine learning model analysis unit 160) in real situations.

On the other hand, when it is a section where EEG signal analysis is requested rather than machine learning model training, the machine learning model analysis unit 160 receives the EEG signal for analysis through the reference potential adjustment unit 110, the filtering unit 120, and the signal conversion unit 130, and then inputs the EEG signal to the machine learning model 140 so that an EEG signal from which a noise component is removed is obtained and output through the machine learning model 140 (S60).

As such, because the EEG signal preprocessing device replaces a data inspection procedure and an ICA procedure of an expert in the related art with a machine learning model analysis procedure, the cost and time required for data analysis may be dramatically reduced and real-time analysis may also be performed.

The EEG signal preprocessing device of the present disclosure replaces an existing EEG preprocessing device, and may be widely applied to all diagnostic fields based on EEG signals.

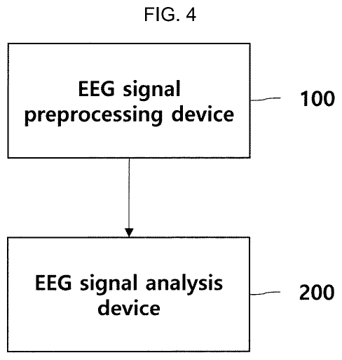

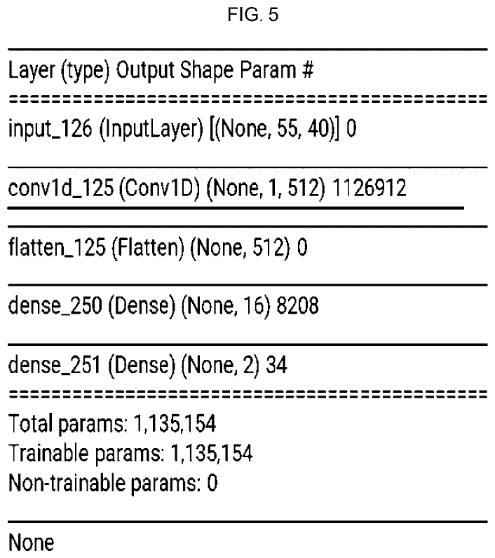

FIGS. 4 and 5 are diagrams illustrating an example of a pain analysis device using an EEG signal preprocessing device according to an embodiment of the present disclosure.

As shown in FIG. 4, a pain analysis device of the present disclosure may include the EEG signal preprocessing device 100 and an EEG signal analysis device 200.

Accordingly, the EEG signal analysis device 200 may receive a high-quality EEG signal from which a noise component is removed through the EEG signal preprocessing device 100, may analyze the high-quality EEG signal, and may determine and notify whether there is pain.

That is, as shown in FIG. 5, the EEG signal preprocessing device 100 may be implemented with a 1D CNN Conv, and the EEG signal analysis device 200 may be implemented with a Flatten layer, a dense layer, and a softmax layer.

In this case, assuming that the number of layers of the 1D CNN is layer_n, data in the form of layer_n×1 is obtained through the 1D CNN, and is flattened into 1D. A pain probability corresponding to an input signal is classified by applying the dense layer and the softmax layer.

While the preferred embodiments of the present disclosure have been shown and described, the present disclosure is not limited to the specific embodiments described above, various modifications may be made by one of ordinary skill in the art to which the present disclosure pertains without departing from the gist of the present disclosure as defined by the claims, and these modifications should not be individually understood from the technical feature or prospect of the present disclosure.

1. An electroencephalogram (EEG) signal preprocessing device comprising:
a reference potential adjustment unit configured to remove a common noise component by adjusting a reference potential of an EEG signal;
a filtering unit configured to filter a frequency band of the EEG signal;
a signal conversion unit configured to convert the EEG signal from a time domain signal into a frequency domain signal;
a machine learning model implemented with a one-dimensional (1D) convolutional neural network (CNN) in which a number of layers is the same as a number of channels of the EEG signal;
a machine learning model training unit configured to generate a plurality of training data having the EEG signal as an input condition and an EEG signal from which a noise component is removed as an output condition, and then perform supervised learning on the machine learning model through the plurality of training data; and
a machine learning model analysis unit configured to, when EEG signal preprocessing is requested, obtain and output an EEG signal from which a noise component corresponding to the EEG signal is removed through the machine learning model.

2. The EEG signal preprocessing device of claim 1, wherein the machine learning model training unit is further configured to re-train the machine learning model by using an input signal and an output signal of the machine learning model analysis unit.

3. An electroencephalogram (EEG) signal preprocessing method comprising:
removing a common noise component by adjusting a reference potential of an EEG signal;
filtering a frequency band of the EEG signal;
converting the EEG signal from a time domain signal into a frequency domain signal;
generating a plurality of training data having the EEG signal as an input condition and an EEG signal from which a noise component is removed as an output condition, and then performing supervised learning on a machine learning model through the plurality of training data; and
when EEG signal preprocessing is requested, obtaining and outputting an EEG signal from which a noise component corresponding to the EEG signal is removed through the machine learning model,
wherein the machine learning model is implemented with a one-dimensional (1D) CNN in which a number of layers is the same as a number of channels of the EEG signal.
